(19) United States
(12) Patent Application Publication
(10) Pub. No.: US 2010/0075310 A1
(43) Pub. Date: Mar. 25, 2010

(54) Methods for Microorganism Detection and Identification

(75) Inventors: Syed K. Hasan, Lowell, MA (US); Christopher Bellotti, Lowell, MA (US)

Correspondence Address: SWANSON & BRATSCHUN, L.L.C., 8210 SOUTHPARK TERRACE, LITTLETON, CO, 80120, US

(73) Assignee: IMMUNOTREX CORPORATION, Lowell, MA (US)

(21) Appl. No.: 12/426,470

(22) Filed: Apr. 20, 2009

Related U.S. Application Data
(63) Continuation of application No. 11/612,221, filed on Dec. 18, 2006.
Application No. 11/612,221 is a continuation of application No. 11/381,692, filed on May 4, 2006, now abandoned.
(60) Provisional application No. 60/677,951, filed on May 4, 2005.

(51) Int. Cl. C12Q 1/68

(52) U.S. Cl. 435/6

(57) ABSTRACT

Methods for detecting and identifying microorganisms in a biologic sample are provided. The methods utilize identifying rRNA from microorganisms to both show the presence and identity for the majority of infectious agents present in clinical samples. The methods are preferably adapted for use in a clinical setting.

### CROSS-REFERENCE TO RELATED APPLICATIONS

This application claims priority to U.S. patent application Ser. No. 11/612,221 entitled “Methods for Microorganism Detection and Identification”, filed Dec. 18, 2006, U.S. patent application Ser. No. 11/381,692 entitled “Methods For Microorganism Detection and Identification”, filed May 4, 2006, and U.S. Provisional Patent Application No. 60/677,951 entitled “Methods and Device For Microorganism Detection and Identification”, filed May 4, 2005, each of which are incorporated by reference herein.

### BACKGROUND

a. Field

The present invention relates generally to the field of microorganism detection and identification, and more specifically to methods for the detection and identification of microorganisms through a microorganism's isolated ribosomal RNA (rRNA).

b. Background

Bacteria, as well as other microorganism, i.e., infections agents, are still a major cause of disease and death in the world, especially in developing countries where malnutrition and poor hygiene exacerbate the problem. Determining the presence and identity of an infectious agent in a timely manner is crucial for minimizing the damage caused by the agent, where a timely diagnosis and treatment may mean the difference between weeks of illness and possibly death and a few days of discomfort.

Typically, infectious materials can be located in the blood, urine, spinal fluid, etc., i.e., biological samples, of an infected individual. Clinical testing on such biological samples are presently performed through culturing techniques and other methods that take anywhere from twenty four to forty eight hours. In addition, present clinical detection and identification techniques have varied sensitivities and accuracies, often requiring repetitive testing over a course of several days.

Against this backdrop the present invention has been developed.

### BRIEF SUMMARY

The present invention provides methods and compositions for detecting and identifying infectious agents in a biologic sample. Methods include providing a sample having the potential infectious agent to a lysis solution; separating any rRNA from lysed infectious agents; isolating the rRNA and performing polymerase chain reaction (PCR) on the sample (for example, using reverse transciptase (RT) RT-PCR to produce corresponding cDNA and amplifying the cDNA using standard PCR techniques). Amplified rRNA is detected on, for example, a DNA chip fabricated to recognize target rRNA sequences from target bacterium, or via other like technique, that results in a diagnostic result. DNA chips are typically fabricated to have at least one immobilized nucleic acid target sequence from a eubacterial and preferably from a pathogenic bacteria of interest. Results can then utilized to provide a best case treatment for the individual who provided the sample.

In one aspect of the invention, the methods of the invention transform laboratory processes into timely and cost effective results, beneficial to the patient and health care provider. This is particularly true in the diagnostic assay situation.

These and various other features and advantages of the invention will be apparent from a reading of the following detailed description and a review of the appended claims.

### BRIEF DESCRIPTION OF THE DRAWINGS

FIG. 1 is a flow diagram illustrating one process for practicing the present invention.

### DETAILED DESCRIPTION

### Definitions

The following definitions are provided to facilitate understanding of certain terms used frequently herein and are not meant to limit the scope of the present disclosure.

As used herein, the term “amplification” is defined as the production of additional copies of a nucleic acid sequence or molecule and is generally carried out using a technique such as polymerase chain reaction (PCR) or other like techniques (see, e.g., Dieffenbach C. W. and G. S. Dveksler (1995) PCR Primer, A Laboratory Manual, Cold Spring Harbor Press, Plainview, N.Y.). With regard to PCR, references U.S. Pat. No. 4,683,202 and U.S. Pat. No. 4,683,195 are hereby incorporated by reference.

As used herein, the terms “nucleic acid,” “nucleic acid molecule” or “NA” refers to the phosphate ester polymeric form of either deoxyribonucleosides (deoxyadenosine, deoxyguanosine, deoxythymidine, or deoxycytidine), ribonucleosides (adenosine, guanosine, uridine or cytidine), or any phosphoester analogs thereof (e.g., phosphorothioates, thioesters). Double-stranded DNA-DNA, DNA-RNA and RNA-RNA helices are included within the definition. The term “nucleotide” includes both individual units of deoxyribonucleic acid and ribonucleic acid as well as nucleoside and nucleotide analog, and modified nucleotides such as labeled nucleotides. In addition, nucleotide includes non-naturally occurring analog structures, such as those in which the sugar, phosphate, and/or base units are absent or replaced by other chemical structures. Thus, the term nucleotide encompasses individual peptide nucleic acid (PNA)(Nielsen et al., Bioconjug. Chem. (1994) 5(1):3-7) and locked nucleic acid (LNA) (Braasch and Corey, Chem. Biol. (2001) 8(1):1-7), for example.

As used herein, “nucleic acid sequence” refers to the order of sequence of nucleotides along a strand of nucleic acid. In some cases, the order of these nucleotides may determine the order of the amino acids along a corresponding polypeptide chain. The nucleotide sequence thus codes for the amino acid sequence. The nucleic acid sequence may be single-stranded or double-stranded, as specified, or contain portions of both double-stranded and single-stranded sequences. The nucleic acid sequence may be composed of DNA, (e.g., genomic, cDNA) RNA, or hybrid, where the sequences comprise any combination of deoxyribo- and ribonucleotides, and any combination of bases or analogs thereof, including uracil, adenine, thymine, cytosine, guanine, isosine, xanthine hypoxathanine, isocytosine, isoguanine, etc.

As used herein, “some portion of rRNA” refers to an amount of rRNA that provides enough material to amplify under either standard PCR methods or methods devoted to real time PCR. Note that rRNA is typically amplified into cDNA using reverse transcriptase prior to or during PCR reactions.

As used herein, “real time PCR” refers to a method(s) for continuously measuring amplification products. Aspects of real time PCR include measurements of amplified nucleic acid during the amplification reaction using a detectable target probe. Typical probes are fluorescent. Various aspects of real time PCR have been described in detail elsewhere, the following references are provided, which are incorporated by reference herein: Lo Y. M. D. et al., Am. J. Hum. Genet. 1998; 62:768 75; Heid C. A. et al., Genome. Res. 1996; 6:986-994; Luthra R et al., Am. J. Pathol. 1998; 153:63-68; Holland et al., Proc. Natl. Acad. Sci. USA 1991; 88:7276-7280.

As used herein, “ribosomal RNA” or “rRNA” refers to the RNA that is the primary constituent of ribosomes. Like other forms of RNA, rRNA is transcribed from DNA, and makes up the majority of the RNA found in a typical cell. In general, there are two mitochondrial rRNA molecules (23S and 16S) and four types of cytoplasmic rRNA (28S, 5.8S, 5S and 18S). For purposes of the present disclosure, rRNA can include the 16S-23S rRNA interspace regions.

### Methods for Identifying Pathogenic Bacteria

In one embodiment, methods are provided for the sensitive molecular diagnostic detection of pathogenic bacteria. Detection methods of the invention rely upon identification of signature rRNA sequences found in specific eubacteria groups. In some embodiments the detection methods are performed on clinical samples.

Ribosomal RNA (rRNA) is highly conserved among eubacteria. However, progressive accumulation of mutations in rRNA sequences has introduced a considerable degree of variability and has subsequently given rise to subsequences within the rRNA gene, which serve as signature sequences for specific eubacteria groups (Kotilainen et al., Jr of Clinical Microbiology (1998) 38(8) 2205-2209; Tseng et al., Clinical Chemistry (2003) No. 2 306-309; Lane et al. PNAS (1985) No. 20 6955-6959, Mokdad et al., Nucleic Acids Res. (2006) 34(5) 1326-1341, all of which are incorporated herein by reference). These signature sequences are used in the present invention for identification of microorganisms in a sample.

In one embodiment, these signature sequences in the rRNA of target eubacteria are amplified into cDNA using reverse transcriptase, or other like enzyme, and then used to fabricate DNA chips as is known in the art. The cDNA containing chip is then used as a platform for identification of rRNA sequences from the potential bacteria contaminates in a sample, and preferably in a clinical sample. The fabrication of cDNA containing chips is known in the art.

Samples for testing within the context of the present invention include any material suspected of having a target bacterium of interest. Typically, samples for purpose of the present invention include biologic samples and in preferred embodiments clinical samples.

Samples of the present invention are lysed under appropriate lysing conditions with an appropriate lysis solution. Compositions of lysis solutions are well known in the art. Lysing solutions result in the release of nucleic acid, including rRNA, from cells, and in particular, are used under conditions that prevent or limit RNA degradation. Released rRNA in a sample is then isolated using centrifugation or other like technique. Isolated rRNA is then amplified into corresponding DNA or other nucleic acid using, for example, reverse transcriptase. The target or identifier sequences from the rRNA that match the cDNA chip sequences are then amplified using standard PCR methods (primers appropriate for each target bacterium) and detected on the before described cDNA chip. In this way the amplified nucleic acid from the extracted rRNA is compared to the known rRNA on the cDNA chip. Positive and negative results would be known in the context of the present invention by one of skill in the art.

Detection methods and protocols for fabricated cDNA chips are known in the art.

In another embodiment, methods are provided that rely on real-time PCR for broad-range amplification and detection of bacterial DNA sequences. This method is particularly useful in clinical applications. As such, isolated rRNA from samples are amplified in real time using real time PCR methods and eubacteria probes of interest. This method would not require a corresponding cDNA chip, but rather a fluorescent or other like probe and use of a standard real time PCR cycler (Roche Light Cycler, Perkin-Elmer Taqman 7700, etc).

In an alternative embodiment, highly specific virulence factors from different bacteria are used to develop antibodies (monoclonal or polyclonal). These antibodies are then arrayed on a slide and used to detect bacteria in a sample. Conversely, virulence factors can be immobilized onto a slide and tested against blood samples of patients, if the patient blood has antibody formed from that particular infection, the antibody will be bound to the slide. Elisa or other immuno-based technique can be used to visualize the result.

Note that for purposes of the present invention, the term “antibody” is used in the broadest sense and specifically covers monoclonal antibodies, polyclonal antibodies, multispecific antibodies, e.g., bispecific antibodies, chimeric antibodies, humanized antibodies, fully synthetic antibodies and antibody fragments so long as they exhibit the desired biologic activity, i.e., binding specificity.

The production of antibodies against virulence factors can be performed by methods known in the art, for example, I. Lefkovits, Ed., (1996) Immunology Methods Manual Academic Press, Inc., San Diego, Calif. (incorporated herein by reference in its entirety). In typical embodiments the antibody is a monoclonal antibody. Hybridoma cell lines that produce the monoclonal antibodies of the present invention are typically produced by a fusion of an immortalized cell line with a B-lymphocyte and cells selected for antibody production that have affinity against the virulence factor of interest, for example, a virulence factor specific to Salmonella.

A single DNA or protein chip that is so designed could have representative rRNA targets (complete gene sequence/partial conserved sequence in form of oligonucleotides) or virulence factors from all pathogenic bacteria known to occur in that clinical sample in disease conditions and would thus provide a comprehensive and exhaustive method for the detection of the same. Note that single rRNA targets could also be used to test samples for specific infectious agents, e.g., testing a blood sample for Pneumococcus.

As an illustrative example, a DNA chip designed to identify enteric pathogens would have representative target molecules from a wide group of bacteria such as Escherichia, Salmonella, Shigella, Vibrio, Yersinia, Bacillus, Clostridium, Campylobacter, etc. Similarly a chip designed to identify the causative agent of bacterial meningitis from the CSF of patients would have rRNA genes/antibodies to virulence factors from a number of organisms such as Neisseria, Mycobacterium, Haemophilus influenzae, Pneumococcus. Alternatively, real time PCR could be performed on the isolated rRNA from samples (after reverse transcription into cDNA) and probed with enteric pathogen specific probe(s).

There are several advantages that are inherent with the use of such a molecular approach for diagnostic purposes. The first most obvious would be the speed of diagnosis which would be far lesser that conventional methods that require culturing of pathogen. This, in addition to biochemical testing of the isolate, requires a minimum period of 2-3 days. For certain bacteria such as members of the genus Mycobacterium an even longer growth period is required. Besides certain pathogenic bacteria are non-culturable on ordinary lab media and require certain highly specialized media and long incubations for their growth. In such cases, where definitive knowledge regarding the causal pathogen is lacking, a broad-spectrum antibacterial treatment regime often has to be prescribed to treat the disease in question. Pathogens in clinical samples may often be non-viable which would lead to an inability to culture them. The molecular method outlined above would provide a conclusive diagnosis within a matter of few hours since it is based upon nucleic acid/protein present in pathogens within the samples and not their growth characteristics. This method would also eliminate the need for pathogens within a sample to be viable. Such a rapid diagnosis in turn would help clinicians devise an effective treatment strategy specifically targeted against the pathogen in question thereby eliminating the potentially harmful/undesired side effects of a broad spectrum treatment. Such a method is also sensitive. An added advantage of this diagnostic method is the minimal volume of sample required because of its inherent high sensitivity and its handiness and simplicity of operation.

The procedure involved using the present invention would require minimum pretreatment of the clinical sample, requiring it to be concentrated and processed for isolating DNA/protein for its application to the DNA/protein chip. The actual visualization of DNA-DNA hybridization in a DNA based microarray or antigen (virulence factor)/antibody on a protein chip could be done by use of chemiluminescent technology. Other advantages such a method would have over conventional diagnostic methods is specificity, sensitivity and rapidity of detection. Such testing would eventually offer more specific treatments

An embodiment of the invention includes adhering to the concepts listed herein:

Each functional module

The operating scenario

The operator interface

The control system

The physical structure

The physical appearance

The design engineering effort of embodiments of the present invention will optimize between creating new items necessary to perform specific functions and selecting suitable purchase items to be integrated “as is” or modified, based on the economics of building one demonstrator only. It is important to note that entirely different considerations, designs, and economics apply to very high numbers of units built for which high cost manufacturing tooling might reduce substantially the unit costs and even change the appearance. In addition, no Regulatory Agency Approval testing is projected for the demonstrator; such testing is absolutely required for any production version.

Having generally described the invention, the same will be more readily understood by reference to the following examples, which are provided by way of illustration and are not intended as limiting.

### EXAMPLES

### Example 1

### Sample Preparation and Bacterial Identification

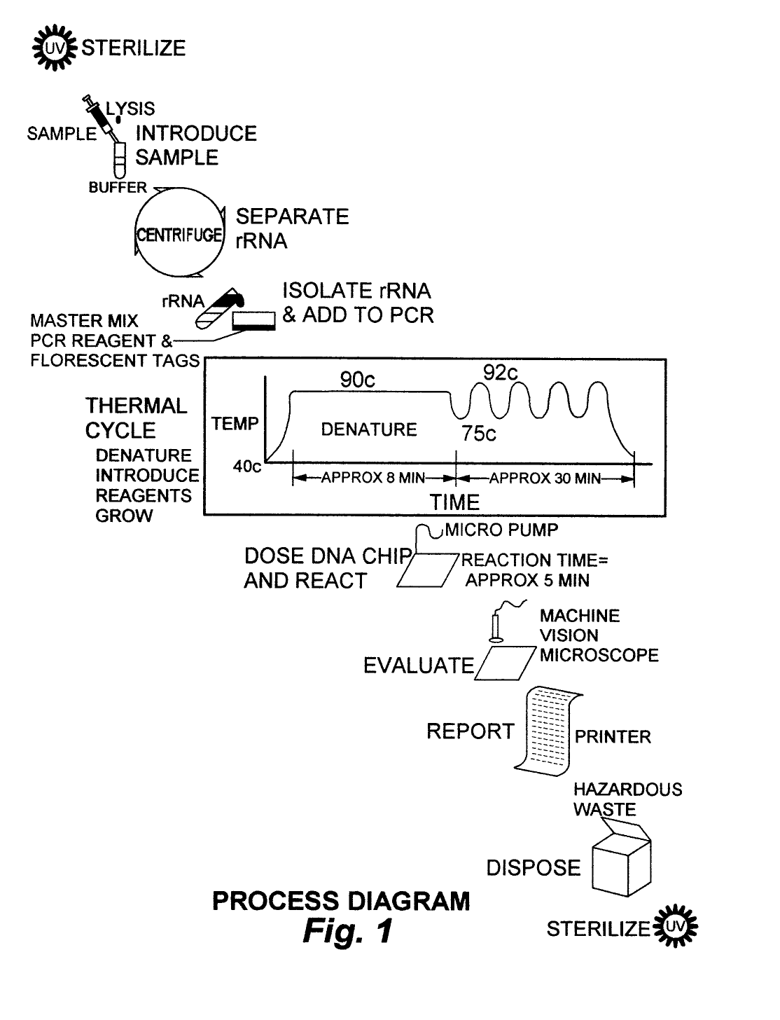

The following example is provided to illustrate the methods of the present invention, performed as follows, and as shown in FIG. 1:
1. This process utilizes bio-reactive samples; contaminating properties of prior samples or residues must be purged or eradicated prior to initiating a new sample.
2. Inject the biologic fluid sample (approximately 1 ml) into a cartridge of protective buffer fluid (approximately 1 ml) at approximately 1:1 ratio.
3. Add Lysis buffer solution (approximately ½ ml) to the mix, agitate to mix and break up.
4. Centrifuge the mixture to separate by density.
5. Retrieve the top 25% (approx ½ ml) and add to a supply of master mix PCR (reagents and fluorescent tags) (approx ½ ml) at approximately 1:1 ratio. Dispose of the residual sample (approx 2 ml) into a bio-contamination retrieval container.
6. Thermally cycle the result (approx 1 ml) while agitating the mix.
7. Dose the result into individual DNA Chip Petri (Approx 25 μl) (DNA chip having been prepared to exhibit a range of one or more cDNA from target pathogenic bacteria or other microorganism).
8. Inspect the reaction optically for color site by site.
9. Report the color matrix (or translate to diagnosis and report diagnosis).
10. Dispose of all residuals, DNA Chip, containers, contaminated utensils, etc. into a bio-contamination retrieval container.
11. Decontaminate all surfaces and utensils remaining.
12. Recommend treatment of patient based on report.

### Example 2

### Manufacturing Cost of an Apparatus Embodiment of the Present Invention

In preferred embodiments, a detection apparatus (ISO 9000 compliant) manufacturing company could proceed in fabricating a device that implements the methods of the present invention. The company would purchase, manufacture, assemble, and test the devices.

### Manufacturing

| $ TBD | 100% Tooling costs presented at Detail Design Review | |
|---|---|---|
| $ TBD | 100% Unit price × Units delivered | |
| Terms: | $ TBD | Tooling Invoiced at Detail Design Review |
| | $ TBD | Unit Invoiced at delivery, net 30 days |

Note that certain conventional techniques of molecular biology, microbiology, and biochemistry are within the skill of the art an may be employed in accordance with this invention. Such techniques are explained fully in the literature, which are all incorporated by reference herein. See, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Vol. I and II (D. N. Glover ed. 1985); Oligonucleotide Synthesis (M. J. Gait ed. 1984); Nucleic Acid Hybridization (B. D. Hames & S. J. Higgins eds. 1985); Immobilized Cells And Enzymes (IRE Press, 1986); B. Perbal, A Practical Guide To Molecular Cloning (1984); F. M. Ausubel et al. (eds), Current Protocols in Molecular Biology, John Wiley & Sons, Inc. (1994).

The following references are useful for providing detail with regard to the isolation of ribosomes and rRNA from target infectious agents, each reference is incorporated by reference herein in its entirety. The references include: Gaudio P A, Gopinathan U, Sangwan V, Hughes T E, Polymerase chain reaction based detection of fungi in infected corneas, Br J Opthalmol. 2002 July; 86(7):755-60; Cloning, sequencing and characterisation of a Listeria monocytogenes gene encoding a fibronectin-binding protein, Gilot P, Jossin Y, Content J, Department of Virology, Pasteur Institute, Brussels, Belgium. pgilot@ben.vub.ac.be; Counting and size classification of active soil bacteria by fluorescence in situ hybridization with an rRNA oligonucleotide probe, Christensen H, Hansen M, Sorensen J, Department of Veterinary Microbiology, Royal Veterinary and Agricultural University, 1870 Frederiksberg C, Denmark. kvlhc@unidhp.uni-c.dk; Direct ribosome isolation from soil to extract bacterial rRNA for community analysis, Felske A, Engelen B, Nubel U, Backhaus H, Biologische Bundesanstalt fur Land-und Forstwirtschaft, Institut fur Biochemie and Pflanzenvirologie, Braunschweig, Germany; A procedure for isolation of pure complexes of acylated and unacylated tRNA(Phe) with ribosomes from Escherichia coli, Chinali G., Dipartimento di Medicina Clinica e Sperimentale, Facolta di Medicina e Chirurgia, Universita di Napoli Federico II, Napoles, Italy; Biochemistry. 1992 Jul. 28; 31(29):6856-64;

Nuclear-encoded chloroplast ribosomal protein L27 of Nicotiana tabacum: cDNA sequence and analysis of mRNA and genes, Elhag G A, Bourque D P, Department of Biochemistry, University of Arizona, Tucson 85721; Dev Biol Stand. 1992; 77:79-85.

Ribosomes as carriers for antigenic determinants of the surface of micro-organisms, Normier G, Pinel A M, Dussourd d'Hinterland L, Wigzell H, Binz H., Centre d'Immunologie et de Biotechnologie Pierre Fabre, Le Puy St-Martin, St-Julien-en-Genevois, France. Methods Enzymol. 1988; 164:188-200;

Isolation and characterization of colicin fragments of bacterial 16S ribosomal RNA, van Knippenberg P H, Heus H A. J Mol Biol. 1979 Mar. 25; 129(1):63-81.

Identification of proteins involved in the peptidyl transferase activity of ribosomes by chemical modification, Dohme F, Fahnestock S R.

The following references are useful for providing detail with regard to the identification of a eubacterial using specific rRNA sites. Each reference is incorporated by reference herein in its entirety.
1) Avian Pathol. 2004 December; 33(6):610-7.
Characterization of streptococci and enterococci associated with septicaemia in broiler parents with a high prevalence of endocarditis.
2) Probl Tuberk Bolezn Legk. 2004; (11):34-7
[Study of collections of Mycobacterium non-tuberculosis by a restriction test of the amplified fragment spacer sequence 16S-23S of ribosomal DNA]
Maiorova A A, Stepanshina V N, Shemiakin I G, Korobova O V.
3) J Clin Microbiol. 2005 March; 43(3):1467-9
First isolation of Bacteroides thetaiotaomicron from a patient with a cholesteatoma and experiencing meningitis.
Feuillet L, Carvajal J, Sudre I, Pelletier J, Thomassin J M, Drancourt M, Cherif A A.
Service de Neurologie Pr ALI CHERIF, Hospital de la Timone, 264 rue Saint Pierre, 13005 Marseille, France
4) J Clin Microbiol. 2005 March; 43(3):1445-7
Comparison of COBAS AMPLICOR Neisseria gonorrhoeae PCR, including confirmation with N. gonorrhoeae-specific 16S rRNA PCR, with traditional culture.
Luijt D S, Bos P A, van Zwet A A, van Voorst Vader P C, Schirm J.
Regional Public Health Laboratory, van Ketwich Verschuurlaan 92, 9721 S W Groningen, The Netherlands
5) J Clin Microbiol. 2005 March; 43(3):1330-40
Systemic disease in Vaal rhebok (Pelea capreolus) caused by mycoplasmas in the mycoides cluster.
Nicolas M M, Stalis I H, Clippinger T L, Busch M, Nordhausen R, Maalouf G, Schrenzel M D.
Arnold and Mabel Beckman Center for Conservation Research, Zoological Society of San Diego, 15600 San Pasqual Valley Rd., Escondido, Calif. 92027, USA
Chadfield M S, Christensen J P, Christensen H, Bisgaard M.
Department of Veterinary Pathobiology, The Royal Veterinary and Agricultural University, Stigbojlen 4, DK-1870

It is understood for purposes of this disclosure that various changes and modifications may be made to the invention that are well within the scope of the invention. Numerous other changes may be made which will readily suggest themselves to those skilled in the art and which are encompassed in the spirit of the invention disclosed herein.

Numerous patents and publications have been cited herein each of which is hereby incorporated by reference for all purposes.

What is claimed is:

1. A method for identifying a microorganism in a biologic sample comprising extracting some portion of the rRNA from the microorganism, amplifying the rRNA to provide an appropriate nucleic acid amplified sample for comparison with other known rRNA from known microorganisms, and comparing the amplified nucleic acid from the extracted rRNA to known rRNA so as to identify the microorganism in the biologic sample.

2. The method of claim 1 wherein the biologic sample is a clinical sample.

3. The method of claim 1 wherein the comparison occurs on a cDNA fabricated chip having target nucleic acids from at least one eubacteria wherein the target nucleic acids correspond to known rRNA from the known microorganism.

4. The method of claim 1 wherein the comparison occurs on a cDNA fabricated chip having target nucleic acids from at least two eubacteria wherein the target nucleic acids correspond to known rRNA from at least two known microorganisms.

5. A method for identifying a microorganism in a biologic sample comprising extracting some portion of the rRNA from the microorganism, amplifying the rRNA to provide an appropriate nucleic acid amplified sample wherein the amplified sample is probed with a probe specific for the microorganism.

6. The method of claim 5 wherein the microorganism specific probe is fluorescent.

7. The method of claim 6 wherein the amplification and identification of the microorganism is performed in a continuous manner.

8. The method of claim 5 further comprising isolating the rRNA form other constituents of the microorganism prior to amplifying the rRNA.
